(19) United States
(12) Patent Application Publication
(10) Pub. No.: US 2006/0040389 A1
(43) Pub. Date: Feb. 23, 2006

(54) Purified compositions of stem cell derived differentiating cells

(76) Inventors: Charles E. Murry, Seattle, WA (US); Michael Alan Laflamme, Seattle, WA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/920,795

(22) Filed: Aug. 17, 2004

(51) Int. Cl. C12N 5/08; G01N 33/567

(52) U.S. Cl. 435/377; 435/366; 435/7.2

(57) ABSTRACT

Viable differentiating cells from in vitro cultures of stem cells are selected for by partial dissociation to provide cell aggregates. Aggregates comprising cells of interest are selected for phenotypic features using methods that substantially maintain the cell to cell contacts in the aggregate.

This invention relates generally to the field of cell biology of cells and differentiation. More specifically, this invention provides methods for the selection of viable differentiating cells from cultures of stem cells.

### BACKGROUND OF THE INVENTION

The growth potential of mammalian embryonic stage cells have been known for many years, but the ability to culture such pluripotent and totipotent stem cells, particularly human stem cells, has only been recently developed. Stem cells have a capacity both for self-renewal and the generation of differentiated cell types. Embryonic stem (ES) cells are derived from cultures of inner cell mass (ICM) cells, and have the property of participating as totipotent cells when placed into host blastocysts. The developmental pathways that endogenous ICM cells or transferred ES cells take to tissue formation and organogenesis has led many to hope that these pathways can be controlled for the development of tissue and organ specific stem cells. The ability of ES cells to grow specialized cells and tissues could provide an unprecedented tool in the clinic, by providing a means for transplantation and repair of damaged muscles, nerves, organs, bones and other tissues. ES cell lines also have a potent ability to replicate in culture, unlike many of the somatic stem cells, which may also be limited to differentiation within specific lineages.

Muscle is one of the largest tissues in the body, and one that can be subjected to severe mechanical and biological stresses. A number of widespread and serious conditions cause necrosis of heart tissue, leading to unrepaired or poorly repaired damage. For example, coronary artery disease, in which the arteries feeding the heart narrow over time, can cause myocardial ischemia, which if allowed to persist, leads to heart muscle death. Another cause of ischemia is myocardial infarction (MI), which occurs when an artery feeding the heart suddenly becomes blocked. This leads to acute ischemia, which again leads to myocardial cell death, or necrosis.

Cardiac tissue death can lead to other heart dysfunctions. If the pumping ability of the heart is reduced, then the heart may remodel to compensate; this remodeling can lead to a degenerative state known as heart failure. Heart failure can also be precipitated by other factors, including valvular heart disease and cardiomyopathy. In certain cases, heart transplantation must be used to repair an ailing heart.

Unlike skeletal muscle, which regenerates from reserve myoblasts called satellite cells, the mammalian heart has a very limited regenerative capacity and, hence, heals by scar formation. The severity and prevalence of these heart diseases has led to great interest in the development of progenitor and stem cell therapy, which could allow the heart to regenerate damaged tissue and ameliorate cardiac injury (see Murry et al. (2002) C.S.H. Symp. Quant. Biol. 67:519-526). For human therapeutic application, a suitable myogenic cell type from either an autologous or appropriately matched allogeneic source may be delivered to the infarcted zone to repopulate the lost myocardium.

A number of different cell types have been considered for such therapies. While some researchers have reported the persistence of markers from somatic cells as diverse as hematopoietic stem cells; mesenchymal stem cells; and even peripheral blood cells; the evidence is, at least thus far, hotly disputed. While improvements can be found in some functional parameters, it does not seem that new myocytes are being produced.

ES cells have the capacity to give rise to all tissues, including those for which no somatic stem cells are known, such as cardiac muscle (see Kehat et al. (2001) J. Clin. Invest. 108:407-414; Mummery et al. (2002) J. Anat. 200:233-242; he et al. (2003) Circ. Res. 93:32-39). ES cells have certain advantages for cardiac repair applications. There are well-defined protocols for the isolation and maintenance of ESCs, and they have a tremendous capacity for in vitro expansion, making them scalable for human applications (Zandstra et al. (2003) Tissue Eng. 9:767-778). Human ESC-derived cardiomyocytes possess the cellular elements required for electromechanical coupling with the host myocardium, such as gap and adherens junctions, and it is therefore expected that, when transplanted, these cells could electrically integrate and contribute to systolic function (see Mummery et al. (2003) Circulation 107:2733-2740). This property represents a significant advantage over other cell types, such as skeletal muscle, which act through modulation of diastolic function (see Reinecke et al. (2000) J. Cell. Biol. 149:731-740; and Reinecke et al. (2002) J. Mol. Cell. Cardiol. 34:241-249).

However, the extraordinary proliferative capacity of ES cells makes it desirable to separate differentiating cells from their pluripotent parents. For example, PCT patent publication WO 01/88104 describes the separation of ES-derived cells into populations of neuronal cells and glial cells by fluroescence-activated cell sorting. The derivation of cardiomyocytes from mouse ES cells is described by Klug et al. (1996) J. Clin. Invest. 98:216-224, and U.S. Pat. No. 6,737,054. The cells were genetically engineered to express a selectable marker controlled by the a-cardiac myosin heavy chain promoter, and could thus be isolated through a drug selection process. Xu et al. (2002) Circ. Res. 91:501, and in U.S. patent application Ser. No 20030022367, describe the differentiation of cardiomyocytes from human ES cells. The differentiated cultures were dissociated and enriched by Percoll density centrifugation to give a population enriched in cardiomyocytes. Fluorescence-activated cell sorting is described by Fleischmann et al. (1998) FEBS Lett. 440:370-376 and Hidaka et al. (2003) Faseb J. 17:740-742. Separation based on physical properties is described by Doevendans et al. (2000) J. Mol. Cell. Cardiol. 32:839-851).

The use of differentiated cells cultured from embryonic stem cells is of great interest for clinical purposes, as well as drug development and research applications. Improved methods of selection to enrich for specific cells of interest are of great value. The present invention addresses these issues.

### SUMMARY OF THE INVENTION

Composition and methods are provided for the enrichment of viable differentiating cells from in vitro cultures of stem cells. The stem cells are cultured in conditions permissive for differentiation and the formation of cellular aggregates, such as embryoid bodies. By partial dissociation, a composition of cell aggregates comprising small numbers of cells is generated, which aggregates have been found to have improved viability relative to dissociated single cells. Aggregates comprising differentiating cells of interest are sorted by a method that substantially maintains the cell to cell contacts in the aggregate. The cells are selected for phenotypic features by positive selection, negative selection, or both. Selections may be performed either sequentially or in parallel. The population of cell aggregates may also be subjected to an initial enrichment step prior to selection.

The sorted cell aggregates are useful in transplantation, for experimental evaluation, and as a source of lineage and cell specific products, including mRNA species useful in identifying genes specifically expressed in these cells, and as targets for the discovery of factors or molecules that can affect them. Sorted cell aggregates may be used, for example, in a method of screening a compound for an effect on the differentiating cells of interest. This involves combining the compound with the cell population of the invention, and then determining any modulatory effect resulting from the compound. This may include examination of the cells for toxicity, metabolic change, or an effect on cell function.

In one embodiment of the invention, the differentiating cells are cells of the cardiomyocyte lineage. Cells of interest include mammalian cells, particularly primate cells and more particularly human cells. These differentiating cells bear cell surface and morphologic markers characteristic of cardiomyocytes, and a proportion of them undergo spontaneous periodic contraction. Highly enriched populations of cardiomyocyte linage cells can be obtained.

In one embodiment of the invention, a population of cell aggregates is provided wherein the aggregates are substantially comprised of cells in the cardiomyocyte lineage. The cardiomyocyte lineage cells may be cardiomyocyte precursor cells, or differentiated cardiomyocytes. Differentiated cardiomyocytes include one or more of primary cardiomyocytes, nodal (pacemaker) cardiomyocytes; conduction cardiomyocytes; and working (contractile) cardiomyocytes, which may be of atrial or ventricular type. A medicament or delivery device containing cell aggregates or cells derived therefrom is provided for treatment of a human or animal body, including formulations for cardiac therapy. Cardiomyocyte lineage cells may be administered to a patient in a method for reconstituting or supplementing contractile and/or pacemaking activity in cardiac tissue.

These and other embodiments of the invention will be apparent from the description that follows. The compositions, methods, and techniques described in this disclosure hold considerable promise for use in diagnostic, drug screening, and therapeutic applications.

### BRIEF DESCRIPTION OF THE FIGURES

FIGS. 1A-1C are an immunohistochemical analysis of cells positively selected for cardiomyocytes.

FIGS. 2A-2B are graphs depicting the increase in numbers of cells expressing (α-MHC after positive and negative selection.

### DETAILED DESCRIPTION OF THE EMBODIMENTS

Viable differentiating cells from in vitro cultures of stem cells are selected for by partially dissociating embryoid bodies or similar structures to provide cell aggregates. Aggregates comprising cells of interest are selected for phenotypic features using methods that substantially maintain the cell to cell contacts in the aggregate.

Methods of interest for selection include bulk systems, such as magnetic separation, panning, etc., where a population of aggregates can be positively selected for the presence of a feature of interest, or negatively selected for the absence of a feature of interest. Sequential sorting methods, e.g. with a particle sorter, may also be employed, provided that cell to cell contacts are maintained.

In one embodiment of the invention, the selection methods of the invention are combined with other enrichment methods, including genetic selection (Klug et al. (1996) J. Clin. Invest. 98:216-224; U.S. Pat. No. 6,737,054); density separation (Xu et al. (2002) Circ. Res. 91:501; U.S. patent application Ser. No. 20030022367); separation based on physical properties (Doevendans et al. (2000) J. Mol. Cell. Cardiol. 32:839-851); and the like. Each the references are herein specifically incorporated by reference for methods of enriching for ES cell derived cardiomyocytes. Alternatively, benefits are provided by practice of the invention in the absence of genetic manipulation of the cells.

Markers for selection include, without limitation, biomolecules present on the cell surface. Such markers include markers for positive selection, which are present on the differentiating cells of interest; and markers for negative selection, which are absent on the differentiating cells of interest, but which typically are present on other cells present in embryoid bodies, e.g. ES cells, endodermal cells, fibroblasts, etc.

Stem cells and cultures thereof. Pluripotent stem cells are cells derived from any kind of tissue (usually embryonic tissue such as fetal or pre-fetal tissue), which stem cells have the characteristic of being capable under appropriate conditions of producing progeny of different cell types that are derivatives of all of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)).

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-I-60, TRA-1-81, and alkaline phosphatase. The globo-series glycolipid GL7, which carries the SSEA-4 epitope, is formed by the addition of sialic acid to the globo-series glycolipid Gb5, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

Culture conditions of interest provide an environment permissive for differentiation, in which stem cells will proliferate, differentiate, or mature in vitro. Such conditions may also be referred to as differentiative conditions. Features of the environment include the medium in which the cells are cultured, any growth factors or differentiation-inducing factors that may be present, and a supporting structure (such as a substrate on a solid surface) if present. Differentiation may be initiated by formation of embryoid bodies (EB), or similar structures. For example, EB can result from overgrowth of a donor cell culture, or by culturing ES cells in suspension in culture vessels having a substrate with low adhesion properties.

In one embodiment of the invention, embryoid bodies are formed by harvesting ES cells with brief protease digestion, and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after around about 7 days to around about 4 weeks.

Optionally, cardiotropic factors are included, as described in U.S. patent application Ser. No. 20030022367, are added to the culture. Such factors may include nucleotide analogs that affect DNA methylation and alter expression of cardiomyocyte-related genes; TGF-β ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFα, and products of the cripto gene; antibodies and peptidomimetics with agonist activity for the same receptors, cells secreting such factors, and the like.

Differentiating Cells. In the context of cell ontogeny, the adjective “differentiated”, or “differentiating” is a relative term. A “differentiated cell” is a cell that has progressed further down the developmental pathway than the cell it is being compared with. Thus, embryonic stem cells can differentiate to lineage-restricted precursor cells (such as a mesodermal stem cell), which in turn can differentiate into other types of precursor cells further down the pathway (such as an cardiomyocyte precursor), and then to an end-stage differentiated cell, which plays a characteristic role in a certain tissue type, and may or may not retain the capacity to proliferate further.

The potential of ES cells to give rise to all differentiated cells provides a means of giving rose to any mammalian cell type, and so a very wide range of culture conditions may be used to induce differentiation, and a wide range of markers may be used for selection. One of skill in the art will be able to select markers appropriate for the desired cell type.

Among the differentiated cells of interest are cells not readily grown from somatic stem cells, or cells that may be required in large numbers and hence are not readily produced in useful quantities by somatic stem cells. Such cells may include, without limitation, neural cells, pancreatic islet cells, hematopoietic cells, and cardiac muscle cells (which are described in detail in the following section).

For example, NCAM may be used as a marker for the selection of aggregates comprising neural lineage cells, inter alia (see Kawasaki et al. (2002) PNAS 99:1580-1585). Neuronal subpopulations can be derived from in vitro differentiation of embryonic stem (ES) cells by treatment of embryo-like aggregates with retinoic acid (RA). The cells express Pax-6, a protein expressed by ventral central nervous system (CNS) progenitors. CNS neuronal subpopulations generated expressed combinations of markers characteristic of somatic motoneurons (Islet-1/2, Lim-3, and HB-9), cranial motoneurons (Islet-1/2 and Phox2b) and interneurons (Lim-1/2 or EN1) (Renoncourt et al. (1998) Mech Dev. 179(1-2):185-97; Harper et al. (2004) PNAS 101(18):7123-8).

Another lineage of interest is pancreatic cells. The pancreas is composed of exocrine and endocrine compartments. The endocrine compartment consists of islets of Langerhans, clusters of four cell types that synthesize peptide hormones: insulin (β cells), glucagon (α cells), somatostatin (γ cells), and pancreatic polypeptide (PP cells). Although the adult pancreas and central nervous system (CNS) have distinct origins and functions, similar mechanisms control the development of both organs. Strategies that induce production of neural cells from ES cells can be adapted for endocrine pancreatic cells. Useful culture conditions include plating EBs into a serum-free medium, expansion in the presence of basic fibroblast growth factor (bFGF), followed by mitogen withdrawal to promote cessation of cell division and differentiation. A B27 supplement and nicotinamide may improve the yield of pancreatic endocrine cells.

Expression of nestin may be useful as a marker for selection of a number of progenitor cells from embryoid bodies. The cells in the pancreatic lineages express GATA-4 and HNF3, as well as markers of pancreatic β cell fate, including the insulin I, insulin II, islet amyloid polypeptide (IAPP), and the glucose transporter-2 (GLUT 2). Glucagon, a marker for the pancreatic α cell, may also induced in differentiated cells. The pancreatic transcription factor PDX-1 is expressed. These ES cell-derived differentiating cells have been shown to self-assemble into structures resembling pancreatic islets both topologically and functionally (Lumelsky et al. (2001) Science 292(5520):1389-94.

Derivation of hematopoietic lineage cells is also of interest. Hematopoietic stem cells and precursors have been well-characterized, and markers for the selection thereof are well known in the art, e.g. CD34, CD90, c-kit, etc. Co-culture of human ES cells with irradiated bone marrow stromal cell lines in the presence of fetal bovine serum (FBS), but without other exogenous cytokines, leads to differentiation of the human ES cells within a matter of days. A portion of these differentiated cells express CD34, the best-defined marker for early hematopoietic cells (Kaufman and Thomson (2002) J Anat. 200(Pt 3):243-8). CD34⁺ and CD34⁺CD38⁻ cells derived from ES cell cultures have a high degree of similarity in the expression of genes associated with hematopoietic differentiation, homing, and engraftment with fresh or cultured bone marrow (Lu et al. (2002) Stem Cells 20(5):428-37

Cardiomyocyte lineage cells. During normal cardiac morphogenesis, the cranio-lateral part of the visceral mesoderm becomes committed to the cardiogenic lineage. Several heart-associated transcription factors, such as Nkx2.5, Hand1, 2, Srf, Tbx5, Gata4, 5, 6 and Mef2c, become expressed in the cardiogenic region. The first possible overt sign of restriction of gastrulating mesodermal cells to the cardiogenic lineage is the expression of the basic helix-loop-helix transcription factor Mesp1. Cardiogenic mesoderm expressing Mesp1 is pluripotent and contains the precursors for the endocardial/endothelial, the epicardial and the myocardial lineages. The cardiomyocytes of the primary heart tube are characterized by low abundance of sarcomeric and sarcoplasmatic reticular transcripts. Myosin light chain (Mlc) 2v is expressed in a part of the tube that gives rise not only to ventricular chamber myocardium, but also to parts of the atrial chambers and to the atrioventricular node. α and β-myosin heavy chain (Mhc), Mc1a, 1v and 2a are initially expressed in the entire heart-tube in gradients, and are later restricted to their compartments.

Morphologically and functionally, the chamber myocardium of the developing atria and ventricles are distinguished from the primary myocardium of the linear heart tube. The chamber myocardium becomes trabeculated, whereas the primary myocardium is smooth and covered with cardiac cushions. The clearest markers that in mammals identify the developing chamber myocardium are the atrial natriuretic factor (Anf) and Cx40 genes, which are not expressed in the myocardium of the primary heart tube. During further development, the smooth-walled dorsal atrial wall (comprising the pulmonary and caval myocardium) as well as the atrial septa are incorporated into the atria. These components do not express Anf, but do express Cx40. A gene that is clearly upregulated in the cardiac chambers is sarco-endoplasmic reticulum Ca2+ ATPase (Serca2a), but because it is also expressed in the primary myocardium it is less suited as a marker for the developing chambers. The functional significance of the chamber program of gene expression is that it allows fast, synchronous contractions.

Based on morphological and electrophysiological criteria, four main phenotypes of cardiomyocytes that arise during development of the mammalian heart can be distinguished: primary cardiomyocytes; nodal cardiomyocytes; conducting cardiomyocytes and working cardiomyocytes. All cardiomyocytes have sarcomeres and a sarcoplasmic reticulum (SR), are coupled by gap junctions, and display automaticity. Cells of the primary heart tube are characterized by high automaticity, low conduction velocity, low contractility, and low SR activity. This phenotype largely persists in nodal cells. In contrast, atrial and ventricular working myocardial cells display virtually no automaticity, are well coupled intercellularly, have well developed sarcomeres, and have a high SR activity. Conducting cells from the atrioventricular bundle, bundle branches and peripheral ventricular conduction system have poorly developed sarcomeres, low SR activity, but are well coupled and display high automaticity.

For α-Mhc, β-Mhc and cardiac Troponin I and slow skeletal Troponin I, developmental transitions have been observed in differentiated ES cell cultures. Expression of Mlc2v and Anf is often used to demarcate ventricular-like and atrial-like cells in ES cell cultures, respectively, although in ESDCs, Anf expression does not exclusively identify atrial cardiomyocytes and may be a general marker of the working myocardial cells.

A “cardiomyocyte precursor” is defined as a cell that is capable (without dedifferentiation or reprogramming) of giving rise to progeny that include cardiomyocytes. Such precursors may express markers typical of the lineage, including, without limitation, cardiac troponin I (cTnI), cardiac troponin T (cTnT), sarcomeric myosin heavy chain (MHC), GATA4, Nkx2.5, N-cadherin, β1-adrenoceptor (β1-AR), ANF, the MEF-2 family of transcription factors, creatine kinase MB (CK-MB), myoglobin, or atrial natriuretic factor (ANF).

Throughout this disclosure, techniques and compositions that refer to “cardiomyocytes” or “cardiomyocyte precursors” can be taken to apply equally to cells at any stage of cardiomyocyte ontogeny without restriction, as defined above, unless otherwise specified. The cells may or may not have the ability to proliferate or exhibit contractile activity.

Certain cells of this invention demonstrate spontaneous periodic contractile activity, whereas others may demonstrate non-spontaneous contractile activity (evoked upon appropriate stimulation). Spontaneous contraction generally means that, when cultured in a suitable tissue culture environment with an appropriate Ca⁺⁺ concentration and electrolyte balance, the cells can be observed to contract in a periodic fashion across one axis of the cell, and then release from contraction, without having to add any additional components to the culture medium. Non-spontaneous contraction may be observed, for example, in the presence of pacemaker cells, or other stimulus.

Markers. The markers for selection of aggregates comprising cells of interest will vary with the specific cells. As described above, a number of well-known markers can be used for positive selection of differentiating cells. Useful markers for positive selection of cardiomyocytes may include, without limitation, one, two or more of NCAM (CD56); HNK-1; L-type calcium channels; cardiac sodium-calcium exchanger; etc. Additional cytoplasmic markers for cardiomyocyte subsets are also of interest, e.g. Mlc2v for ventricular-like working cells; and Anf as a general marker of the working myocardial cells. Markers for pacemaker cells also include HCN2, HCN4, connexin 40, etc.

Markers for negative selection are also of interest, particularly markers that are selectively expressed on ES cells, fibroblasts, epithelial cells, etc. Epithelial cells may be selected for as ApCAM positive. A fibroblast specific selection agent is commercially available from Miltenyi Biotec (see Fearns and Dowdle (1992) Int. J. Cancer 50:621-627 for discussion of the antigen). Markers found on ES cells suitable for negative selection include SSEA-3, SSEA-4, TRA-I-60, TRA-1-81, and alkaline phosphatase.

Specific Binding Member. The term “specific binding member” or “binding member” as used herein refers to a member of a specific binding pair, i.e. two molecules, usually two different molecules, where one of the molecules (i.e., first specific binding member) through chemical or physical means specifically binds to the other molecule (i.e., second specific binding member). The complementary members of a specific binding pair are sometimes referred to as a ligand and receptor; or receptor and counter-receptor. Such specific binding members are useful in positive and negative selection methods. Specific binding pairs of interest include carbohydrates and lectins; complementary nucleotide sequences; peptide ligands and receptor; effector and receptor molecules; hormones and hormone binding protein; enzyme cofactors and enzymes; enzyme inhibitors and enzymes; etc. The specific binding pairs may include analogs, derivatives and fragments of the original specific binding member. For example, a receptor and ligand pair may include peptide fragments, chemically synthesized peptidomimetics, labeled protein, derivatized protein, etc.

Especially useful reagents are antibodies specific for markers present on the desired cells (for positive selection) and undesired cells (for negative selection). Whole antibodies may be used, or fragments, e.g. Fab, F(ab′)₂, light or heavy chain fragments, etc. Such selection antibodies may be polyclonal or monoclonal and are generally commercially available or alternatively, readily produced by techniques known to those skilled in the art. Antibodies selected for use will have a low level of non-specific staining and will usually have an affinity of at least about 100 μM for the antigen.

In one embodiment of the invention, antibodies for selection are coupled to a magnetic reagent, such as a superparamagnetic microparticle, which antibodies may be referred to as “magnetized”. Herein incorporated by reference, Molday (U.S. Pat. No. 4,452,773) describes the preparation of magnetic iron-dextran microparticles and provides a summary describing the various means of preparing particles suitable for attachment to biological materials. A description of polymeric coatings for magnetic particles used in high gradient magnetic separation (HGMS) methods are found in U.S. Pat. No. 5,385,707. Methods to prepare superparamagnetic particles are described in U.S. Pat. No. 4,770,183. The microparticles will usually be less than about 100 nm in diameter, and usually will be greater than about 10 nm in diameter. The exact method for coupling is not critical to the practice of the invention, and a number of alternatives are known in the art. Direct coupling attaches the antibodies to the particles. Indirect coupling can be accomplished by several methods. The antibodies may be coupled to one member of a high affinity binding system, e.g. biotin, and the particles attached to the other member, e.g. avidin. One may also use second stage antibodies that recognize species-specific epitopes of the antibodies, e.g. anti-mouse Ig, anti-rat Ig, etc. Indirect coupling methods allow the use of a single magnetically coupled entity, e.g. antibody, avidin, etc., with a variety of separation antibodies.

### Section of Viable Differentiating Cells

ES cells or cell lines as described above can be propagated continuously in culture, using culture conditions that promote proliferation without promoting differentiation, using methods known in the art. Methods of culture are described, for example, in U.S. patent application Ser. No. 20030190748 (Serum free cultivation of primate embryonic stem cells); U.S. patent application Ser. No. 20040023376 (Method of making embryoid bodies from primate embryonic stem cells); U.S. patent application Ser. No. 20030008392 (Primate embryonic stem cells), each herein incorporated by reference. Conventionally, ES cells are cultured on a layer of feeder cells, typically fibroblasts derived from embryonic or fetal tissue, alternatively cells can be cultured on an extracellular matrix of Matrigel™ or laminin, in medium conditioned by feeder cells or medium supplemented with growth factors such as FGF and SCF (International patent publication WO 01/51616). Under the microscope, ES cells appear with high nuclear/cytoplasmic ratios, prominent nucleoli, and compact colony formation with poorly discernable cell junctions. Differentiation of hES cells in vitro typically results in the loss of these markers (if present) and increased expression of SSEA-1.

Differentiating cells of this invention are obtained by culturing or differentiating stem cells in a growth environment that enriches for cells with the desired phenotype (either by outgrowth of the desired cells, or by inhibition or killing of other cell types). These methods are applicable to many types of stem cells, and for many types of differentiated cells. In one embodiment of the invention, cells are differentiated into cells of the cardiomyocyte lineage, for example as described by U.S. patent application Ser. No. 20030022367.

The culture may optionally comprise agents that enhance differentiation to a specific lineage. For example cardiomyocyte differentiation may be promoting by including cardiotropic agents in the culture, e.g. compounds capable of forming a high energy phosphate bond (such as creatine); an acyl group carrier molecule (such as carnitine); and a cardiomyocyte calcium channel modulator (such as taurine).

The embryoid bodies are harvested at an appropriate stage of development, which may be determined based on the expression of markers and phenotypic characteristics of the desired cell type e.g. at from about 1 to 4 weeks. Cultures may be empirically tested by staining for the presence of the markers of interest, by morphological determination, etc. Where the differentiating cells are cells of the cardiomyocyte lineage, criteria may include spontaneous periodic contractile activity, expression of markers as described above, morphology characteristic of cardiomyocytes, etc.

The embryoid bodies are digested with enzymes, chelators, etc., as known in the art using time, temperature, concentration and selection of reagents that will achieve a partial digestion that leaves aggregates of cells. One of skill in the art can readily perform a simple titration to determine suitable conditions, e.g. using elastase; dispase; collagenase; trypsin; blendzyme; and the like.

The degree of aggregation as referred to herein will be understood to be a mean value, where a normal distribution of sizes will be observed in a population. Aggregates will usually comprise at least about 2 cells, usually at least about 3 cells, more usually at least about 5 cells, and not more than about 50 cells, usually not more than about 40 cells, more usually not more than about 15 cells.

The cells are optionally enriched before or after the positive selection step by drug selection (as described by Klug et al., supra.), panning, density gradient centrifugation, etc. In one method of interest, the composition of cardiomyocytes is enriched by density separation on a discontinuous gradient of Percoll™. The cell suspension is loaded onto a layer of 40.5% Percoll™ (Pharmacia) (approximately 1.05 g/mL) overtop of a layer of 58.5% Percoll™ (approximately 1.075 g/mL). The cells were then centrifuged at 1500 g for 30 min. The cell fractions in the 58.5% layer (fraction IV) are most enriched for cell expressing cardiomyocyte markers.

In another embodiment, a negative selection is performed, where the selection is based on expression of one or more of markers found on ES cells, fibroblasts, epithelial cells, and the like. Selection may utilize panning methods, magnetic particle selection, particle sorter selection, and the like.

For positive or negative selection, separation of the subject cell population utilizes affinity separation to provide a substantially pure population. Techniques for affinity separation may include magnetic separation using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, e.g. plate, or other convenient technique. Any technique may be employed which is not unduly detrimental to the viability of the selected cell aggregates.

Specific binding members, usually antibodies, are added to the suspension of cell aggregates, and incubated for a period of time sufficient to bind the available antigens. The incubation will usually be at least about 2 minutes and usually less than about 30 minutes. It is desirable to have a sufficient concentration of antibodies in the reaction mixture so that the efficiency of the magnetic separation is not limited by lack of antibody. The appropriate concentration is determined by titration.

The suspension of cell aggregates is applied to a separation device. In one embodiment, the separation device is a particle sorter, e.g. as described in U.S. Pat. No. 6,482,652; or as sold by Union Biometrica (COPAS™ systems) for large particle sorting.

In another embodiment, the separation device is a magnetic separation device, and the antibodies are coupled to a magnetic reagent, such as a superparamagnetic microparticle (microparticle). The labeled cells are retained in the magnetic separation device in the presence of a magnetic field, usually at least about 100 mT, more usually at least about 500 mT, usually not more than about 2 T, more usually not more than about 1 T. The source of the magnetic field may be a permanent or electromagnet. After the initial binding, the device may be washed with any suitable physiological buffer to remove unbound cells.

The unbound cells contained in the eluate may be collected as the eluate passes through the separation device. The bound cells, containing the differentiating cells, are released by removing the magnetic field, and eluting in a suitable buffer. The cells may be collected in any appropriate medium. Various media are commercially available and may be used according to the nature of the cells, including dMEM, HBSS, dPBS, RPMI, PBS-EDTA, PBS. Iscove's medium, etc., frequently supplemented with fetal calf serum, BSA, HSA, etc.

Where greater purity is desired, additional separation steps may be performed. The eluted, magnetic fraction may be passed over a second magnetic column to reduce the number of non-specifically bound cells. Higher purity of differentiating cells is also obtained by performing two enrichment steps, using two different differentiating specific separation markers. Alternatively, a multiparameter separation may be performed, by negative selection for ES, or other cells from the sample. The negative selection step may be performed first, followed by the positive selection step, performed essentially as described above, except that the non-magnetic fraction is collected. The enrichment is then performed on the ES cell depleted fraction.

The composition of selected cell aggregates is enriched for the desired differentiating cell type or lineage. Usually at least about 50% of the aggregates will comprise at least one of the selected differentiating cells, more usually at least about 75% of the aggregates, and preferably at least about 90% of the aggregates. Aggregates tend to comprise similar cells, and usually at least about 50% of the total cells in the population will be the selected differentiating cells, more usually at least about 75% of the cells, and preferably at least about 90% of the cells.

The compositions thus obtained have a variety of uses in clinical therapy, research, development, and commercial purposes. For therapeutic purposes, for example, cardiomyocytes and their precursors may be administered to enhance tissue maintenance or repair of cardiac muscle for any perceived need, such as an inborn error in metabolic function, the effect of a disease condition, or the result of significant trauma.

To determine the suitability of cell compositions for therapeutic administration, the cells can first be tested in a suitable animal model. At one level, cells are assessed for their ability to survive and maintain their phenotype in vivo. Cell compositions are administered to immunodeficient animals (such as nude mice, or animals rendered immunodeficient chemically or by irradiation). Tissues are harvested after a period of regrowth, and assessed as to whether the administered cells or progeny thereof are still present.

This can be performed by administering cells that express a detectable label (such as green fluorescent protein, or β-galactosidase); that have been prelabeled (for example, with BrdU or [³H] thymidine), or by subsequent detection of a constitutive cell marker (for example, using human-specific antibody). The presence and phenotype of the administered cells can be assessed by immunohistochemistry or ELISA using human-specific antibody, or by RT-PCR analysis using primers and hybridization conditions that cause amplification to be specific for human polynucleotides, according to published sequence data.

Where the differentiating cells are cells of the cardiomyocyte lineage, suitability can also be determined in an animal model by assessing the degree of cardiac recuperation that ensues from treatment with the differentiating cells of the invention. A number of animal models are available for such testing. For example, hearts can be cryoinjured by placing a precooled aluminum rod in contact with the surface of the anterior left ventricle wall (Murry et al., J. Clin. Invest. 98:2209, 1996; Reinecke et al., Circulation 100:193, 1999; U.S. Pat. No. 6,099,832). In larger animals, cryoinjury can be inflicted by placing a 30-50 mm copper disk probe cooled in liquid N₂ on the anterior wall of the left ventricle for approximately 20 min (Chiu et al., Ann. Thorac. Surg. 60:12, 1995). Infarction can be induced by ligating the left main coronary artery (Li et al., J. Clin. Invest. 100:1991, 1997). Injured sites are treated with cell preparations of this invention, and the heart tissue is examined by histology for the presence of the cells in the damaged area. Cardiac function can be monitored by determining such parameters as left ventricular end-diastolic pressure, developed pressure, rate of pressure rise, and rate of pressure decay.

The differentiated cells may be used for tissue reconstitution or regeneration in a human patient or other subject in need of such treatment. The cells are administered in a manner that permits them to graft or migrate to the intended tissue site and reconstitute or regenerate the functionally deficient area. Special devices are available that are adapted for administering cells capable of reconstituting cardiac function directly to the chambers of the heart, the pericardium, or the interior of the cardiac muscle at the desired location. The cells may be administered to a recipient heart by intracoronary injection, e.g. into the coronary circulation. The cells may also be administered by intramuscular injection into the wall of the heart.

Medical indications for such treatment include treatment of acute and chronic heart conditions of various kinds, such as coronary heart disease, cardiomyopathy, endocarditis, congenital cardiovascular defects, and congestive heart failure. Efficacy of treatment can be monitored by clinically accepted criteria, such as reduction in area occupied by scar tissue or revascularization of scar tissue, and in the frequency and severity of angina; or an improvement in developed pressure, systolic pressure, end diastolic pressure, patient mobility, and quality of life.

The differentiating cells may be administered in any physiologically acceptable excipient, where the cells may find an appropriate site for regeneration and differentiation. The cells may be introduced by injection, catheter, or the like. The cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by use of growth factors and/or feeder cells associated with progenitor cell proliferation and differentiation.

The cells of this invention can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types, especially endothelial cells.

Cells may be genetically altered in order to introduce genes useful in the differentiated cell, e.g. repair of a genetic defect in an individual, selectable marker, etc., or genes useful in selection against undifferentiated ES cells. Cells may also be genetically modified to enhance survival, control proliferation, and the like. Cells may be genetically altering by transfection or transduction with a suitable vector, homologous recombination, or other appropriate technique, so that they express a gene of interest. In one embodiment, cells are transfected with genes encoding a telomerase catalytic component (TERT), typically under a heterologous promoter that increases telomerase expression beyond what occurs under the endogenous promoter, (see International Patent Application WO 98/14592). In other embodiments, a selectable marker is introduced, to provide for greater purity of the desired differentiating cell. Cells may be genetically altered using vector containing supernatants over a 8-16 h period, and then exchanged into growth medium for 1-2 days. Genetically altered cells are selected using a drug selection agent such as puromycin, G418, or blasticidin, and then recultured.

The cells of this invention can also be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in the differentiated cell type. Of particular interest are cells that are genetically altered to express one or more growth factors of various types, cardiotropic factors such as atrial natriuretic factor, cripto, and cardiac transcription regulation factors, such as GATA-4, Nkx2.5, and MEF2-C.

Many vectors useful for transferring exogenous genes into target mammalian cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, etc. For modification of stem cells, lentiviral vectors are preferred. Lentiviral vectors such as those based on HIV or FIV gag sequences can be used to transfect non-dividing cells, such as the resting phase of human stem cells (see Uchida et al. (1998) P.N.A.S. 95(20): 11939-44).

Combinations of retroviruses and an appropriate packaging line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g. MMLV, are capable of infecting most murine and rat cell types. Ecotropic packaging cell lines include BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse. Amphotropic packaging cell lines include PA12 (Miller et al. (1985) Mol. Cell. Biol. 5:431-437); PA317 (Miller et al. (1986) Mol. Cell. Biol. 6:2895-2902) GRIP (Danos et al. (1988) PNAS 85:6460-6464). Retroviruses packaged with xenotropic envelope protein, e.g. AKR env, are capable of infecting most mammalian cell types, except murine cells.

The vectors may include genes that must later be removed, e.g. using a recombinase system such as Cre/Lox, or the cells that express them destroyed, e.g. by including genes that allow selective toxicity such as herpesvirus TK, bcl-xs, etc.

Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

The cells of this invention can be used to prepare a cDNA library relatively uncontaminated with cDNA preferentially expressed in cells from other lineages. For example, cardiomyocytes are collected by centrifugation at 1000 rpm for 5 min, and then mRNA is prepared from the pellet by standard techniques (Sambrook et al., supra). After reverse transcribing into cDNA, the preparation can be subtracted with cDNA from undifferentiated ES cells, other progenitor cells, or end-stage cells from the cardiomyocyte or any other developmental pathway.

The differentiated cells of this invention can also be used to prepare antibodies that are specific for markers of cardiomyocytes and their precursors. Polyclonal antibodies can be prepared by injecting a vertebrate animal with cells of this invention in an immunogenic form. Production of monoclonal antibodies is described in such standard references as U.S. Pat. Nos. 4,491,632, 4,472,500 and 4,444,887, and Methods in Enzymology 73B:3 (1981). Specific antibody molecules can also be produced by contacting a library of immunocompetent cells or viral particles with the target antigen, and growing out positively selected clones. See Marks et al., New Eng. J. Med. 335:730, 1996, and McGuiness et al., Nature Biotechnol. 14:1449, 1996. A further alternative is reassembly of random DNA fragments into antibody encoding regions, as described in EP patent application 1,094,108 A.

The antibodies in turn can be used to identify or rescue cells of a desired phenotype from a mixed cell population, for purposes such as costaining during immunodiagnosis using tissue samples, and isolating precursor cells from terminally differentiated cardiomyocytes and cells of other lineages.

Of particular interest is the examination of gene expression in the differentiating of the invention. The expressed set of genes may be compared against other subsets of cells, against ES cells, against adult heart tissue, and the like, as known in the art. Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A+ mRNA. One of skill in the art can readily use these methods to determine differences in the size or amount of mRNA transcripts between two samples.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). In short, SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680.

Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

Methods for collection of data from hybridization of samples with an array are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

In another screening method, the test sample is assayed for the level of polypeptide of interest. Diagnosis can be accomplished using any of a number of methods to determine the absence or presence or altered amounts of a differentially expressed polypeptide in the test sample. For example, detection can utilize staining of cells or histological sections (e.g., from a biopsy sample) with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.) The absence or presence of antibody binding can be determined by various methods, including flow cytometry of dissociated cells, microscopy, radiography, scintillation counting, etc. Any suitable alternative methods can of qualitative or quantitative detection of levels or amounts of differentially expressed polypeptide can be used, for example ELISA, western blot, immunoprecipitation, radioimmunoassay, etc.

The cells are also useful for in vitro assays and screening to detect factors that are active on differentiating cells, including cells of the cardiomyocyte lineage. Of particular interest are screening assays for agents that are active on human cells. A wide variety of assays may be used for this purpose, including immunoassays for protein binding; determination of cell growth, differentiation and functional activity; production of factors; and the like.

In screening assays for biologically active agents, viruses, etc. the subject cells, usually a culture comprising the subject cells, is contacted with the agent of interest, and the effect of the agent assessed by monitoring output parameters, such as expression of markers, cell viability, and the like. The cells may be freshly isolated, cultured, genetically altered as described above, or the like. The cells may be environmentally induced variants of clonal cultures: e.g. split into independent cultures and grown under distinct conditions, for example with or without virus; in the presence or absence of other cytokines or combinations thereof. The manner in which cells respond to an agent, particularly a pharmacologic agent, including the timing of responses, is an important reflection of the physiologic state of the cell.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

In addition to complex biological agents, such as viruses, candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 :l to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of the selected markers. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

The composition may optionally be packaged in a suitable container with written instructions for a desired purpose, such as the reconstitution of cardiomyocyte cell function to improve some abnormality of the cardiac muscle.

For further elaboration of general techniques useful in the practice of this invention, the practitioner can refer to standard textbooks and reviews in cell biology, tissue culture, embryology, and cardiophysiology. With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998). With respect to the culture of heart cells, standard references include The Heart Cell in Culture (A. Pinson ed., CRC Press 1987), Isolated Adult Cardiomyocytes (Vols. I & II, Piper & Isenberg eds, CRC Press 1989), Heart Development (Harvey & Rosenthal, Academic Press 1998), 1 Left my Heart in San Francisco (T. Bennet, Sony Records 1990); and Gone with the Wnt (M. Mitchell, Scribner 1996).

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLES

### Example 1

A perceived need for a high degree of cardiac purity motivated the development of methods for cardiac enrichment with magnetic cell sorting (hereafter, “MACS”) detailed below. MACS technology was selected to achieve cell separation with appropriate surface markers instead of fluorescence-activated cell sorting (“FACS”) because FACS requires a preparation of a single-cell suspension for success, as clustered cells are typically rejected or otherwise inappropriately separated by the device. Most of the viable human ESC-derived cardiomyocytes reside within tightly adherent clumps of 5-50 cells, which cannot be sorted by conventional flow cytometry. Enzymatic and/or physical treatment of these cells to obtain a single-cell suspension resulted in a drop in cell viability, possibly as a result of the tight adherens junctions typical of myocytes in intact cardiac tissue, which typically require aggressive treatment for disruption. In addition, MACS is a comparatively inexpensive and simple-to-execute technique that allows sorting of large numbers of cells in a short period of time.

Methods

Two of the originally derived human ESC lines (the male H1 and female H7 lines, Thomson et al. (1998) Science 282:1145-1147) were maintained in the undifferentiated state using previously detailed feeder-free conditions (Xu et al. (2001) Nat. Biotech. 19:971-974). Differentiation was achieved by methods previously shown to result in substantial cardiomyogenesis (Xu et al. (2002) Circ. Res. 91:501-508).

In brief, embryoid bodies were formed by allowing small clumps of undifferentiated human ESCs to grow in suspension culture, and differentiation was induced by withdrawal of mouse embryonic fibroblast-conditioned medium. After 4 days in suspension culture, embryoid bodies were plated onto gelatin-coated substrates, and the adherent outgrowths were fed every 1-2 days with differentiation medium containing 20% fetal bovine serum (Hyclone). Consistent with prior experience, beating foci were first noticeable after 8-10 days in culture, with maximal numbers of beating clusters observed approximately 3 weeks from the initiation of differentiation. Mature embryoid body outgrowth cultures were then enzymatically-dispersed (Blendzyme4, 0.56 U/ml in PBS at 37° C. for 30 min) and then partially enriched for cardiomyocytes using discontinuous Percoll gradient centrifugation as previously described (Xu et al. (2002), supra.)

Further enrichment via MACS was performed on the cell population isolated from the densest Percoll layer (so-called “fraction IV” cells). Toward this end, fraction IV cells were washed twice in serum-free, low calcium buffer (PBS supplemented with 1 mM EDTA and 0.2% BSA) and then incubated with magnetic-bead conjugated antibody of interest (all at 1:5 v/v for 20 minutes at 4° C. Employed bead-conjugated antibodies (all from Miltenyi Biotech, Auburn, Calif.) were as follows: anti-NCAM (CD56), anti-epithelial cells (clone HEA-125), and anti-fibroblast.

Incubated cells were next washed with buffer and then passed over a Mini-MACS separation column (Miltenyi Biotech) as per manufacturer instructions, with collection of positive- and negatively-selected cells after elution.

Immunohistochemistry and quantitative RT-PCR for cardiac-specific markers were used in order to quantify the degree of enrichment achieved with these interventions. Immunohistochemical studies were performed on both cell pellets (e.g. cell preparations in which cells were concentrated by centrifugation, methanol-fixed, and submitted for tissue processing, paraffin embedding, and routine histologic sections) and plated-out cells (e.g. cell preparations re-cultured for 24 hours in usual differentiation medium on gelatin-coated chamber slides, prior to methanol-fixation and immunostaining). In either instance, subsequent immunostaining was performed as previously described (Reinecke et al. (2002) J. Mol. Cell. Cardiol. 34:241-249), using antibodies recognizing either sarcomeric myosin heavy chain (clone MF-20, Developmental Studies Hybridoma Bank) and sarcomeric actin (clone 5C5, Sigma). Note that, while both antibodies will recognize all striated muscle, numerous independent experiments have shown differentiating cultures at this timepoint to be devoid of skeletal muscle cells, allowing these antibodies to be used as a specific cardiac markers under these conditions.

For analysis by quantitative RT-PCR, 1-2×10⁶ cells were lysed (RNA Easy, Qiagen, Valencia, Calif.) and a relative quantitation of the cardiac-specific transcript α-myosin heavy-chain (α-MHC) as generally described by Xu et al. (2002), supra, using a-MHC specific probes and primers, standardized by the ΔΔCT method.

Results

Enrichment Using Positive Selection for NCAM. NCAM was primarily used as a surface marker for positive-selection MACS separation. One issue has been in terms of scale, that is, the generation of starting cultures with sufficient quantities of cells that useable numbers remain after Percoll fractionation and/or MACS. One way of overcoming this obstacle was to perform the MACS separation on a less pure but correspondingly more numerous cell population. However, the better the starting population, the more successful any subsequent MACS enrichment would be expected to be.

By necessity, the first efforts involved NCAM positive MACS selection on the less-pure, “fraction III” Percoll-enriched cells. After preparation, the initial, starting fraction III and MACS+ fraction III cells populations were then re-plated, cultured for 24 hours, and then fixed and immunostained for sarcomeric actins. The percentage of sarcomeric actin positive cardiac cells in each was then determined by a blinded observer. This analysis indicated that a 4.1±1.7 fold enhancement in the percentage of cardiac cells by NCAM MACS selection (n=3 biological replicates).

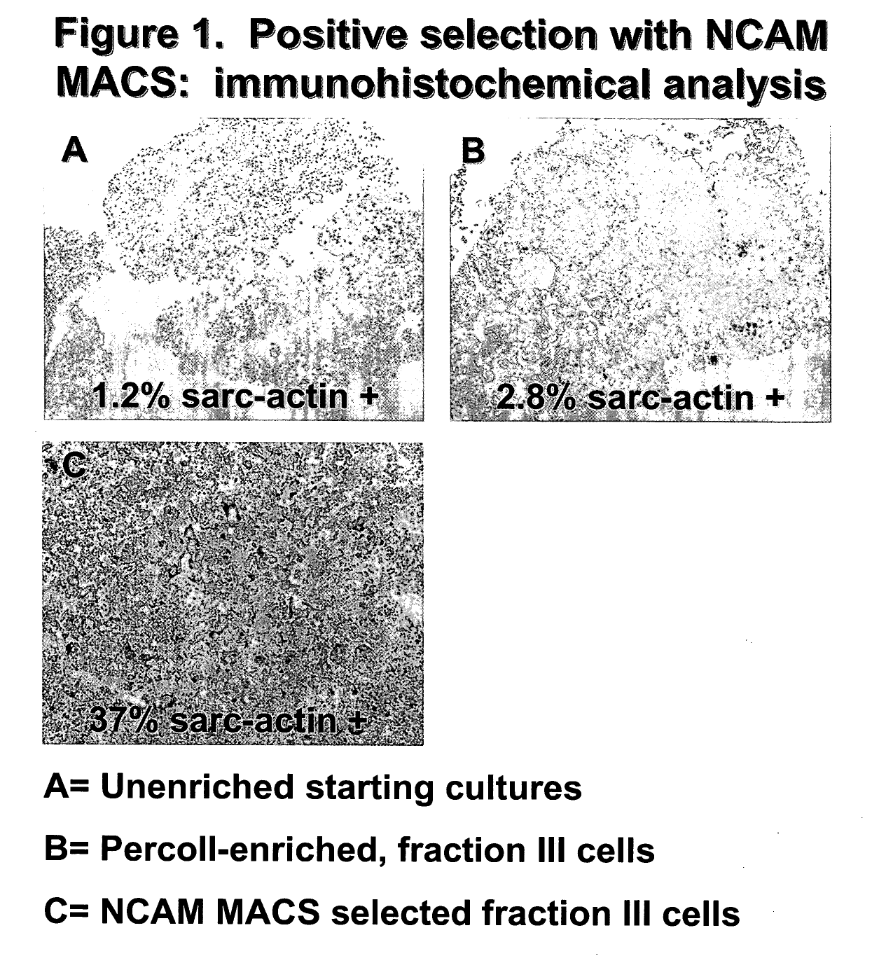

A more recent experiment suggests that the preceding analysis may in fact underestimate the true degree of cardiac enrichment. In this experiment, rather than culturing cells prior to immunohistochemical analysis, the various starting and resultant cell populations were immediately centrifuged down to a pellet, fixed, and then submitted directly for histological embedding and sectioning. This analysis is the “gold-standard” assay in the determination of relative cardiac purity, as it eliminates the confounding variables of plating efficiency, which undoubtedly differs between cardiac and non-cardiac cell types. It is also superior to RT-PCR because of the potential mismatch between purity on a cellular level and by transcript in aggregate. The sectioned cell pellets in this experiment were then immunostained with sarcomeric actins, and the percentage of cells positive for this cardiac marker in each cell population was then determined by a blinded observer. This analysis that the starting population (unfractionated, unenriched embryoid body outgrowth cells) was only 1.2% cardiac, this improving modestly to 2.8% with Percoll fractionation, and, remarkably, to 37% following NCAM MACS. (See FIG. 1A-C).

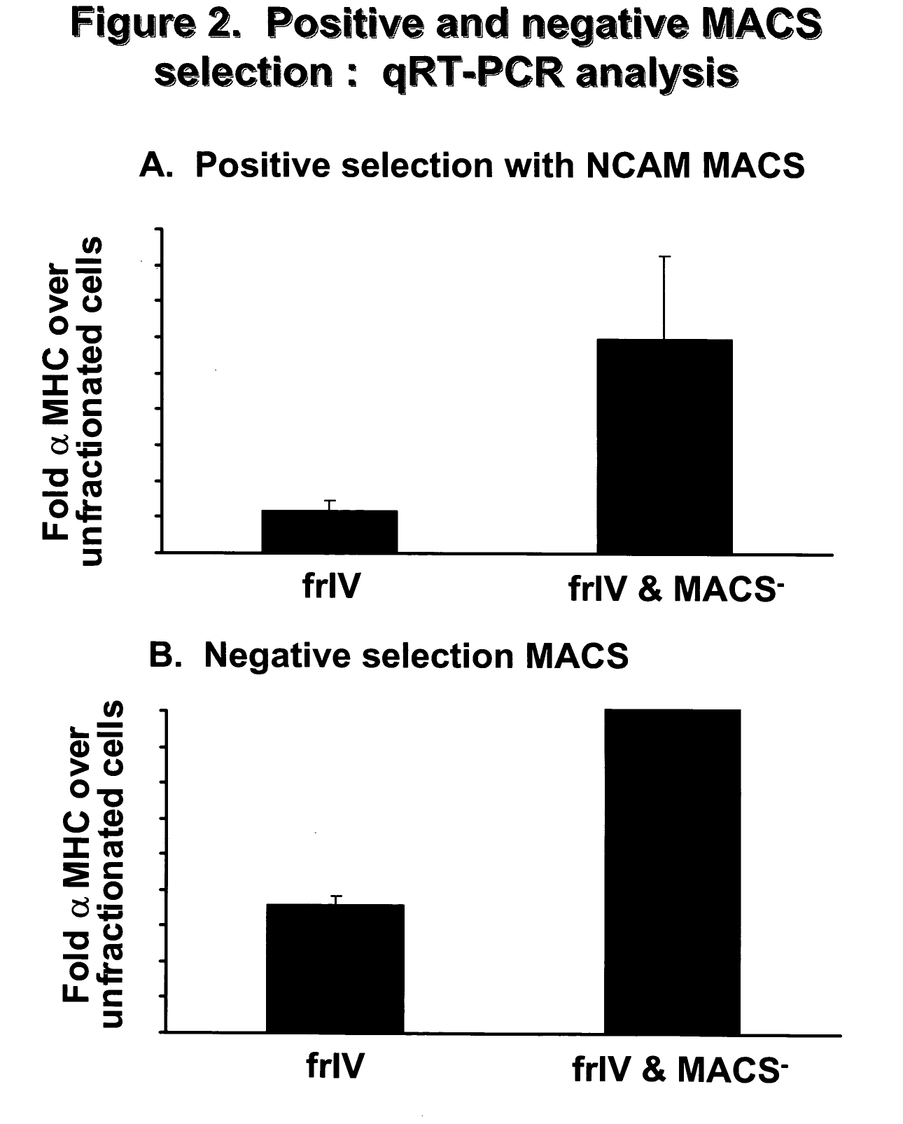

Because of limited numbers of available cells, analysis of the fold-enhancement possible with NCAM MACS on the more-pure, “fraction IV” Percoll-enriched cells has been much more limited. Nonetheless, because transplantation applications necessitate the isolation of the purest population of cells attainable, “fraction IV” cells may be the most useful pre-MACS starting preparation. A large cohort of experiments has been recently performed to test the performance of MACS on fraction IV cells. One recent application of the NCAM MACS protocol on purer fraction IV cells resulted in an approximate six-fold cardiac enrichment, as indexed by quantitative RT-PCR for the cardiac α-MHC transcript (see FIG. 2A).

Enrichment Using Negative Selection. Studies into the degree of enrichment possible with a negative selection strategy are also effective. Three preparations of fraction IV, Percoll-enriched cells were submitted to negative MACS selection, all using a cocktail of magnetic bead-conjugated, anti-epithelial and anti-fibroblast beads. Currently available analysis for degree of cardiac enrichment on the resultant cell populations include quantitative RT-PCR for α-MHC transcript and immunostaining for sarcomeric actins (5C5 clone) on cells plated-out and fixed at 24 hours.

As depicted in FIG. 2B, quantitative RT-PCR for α-MHC indicated a mean approximately five-fold enrichment for the cardiac transcript following depletion (n=3 biological replicates, with the mean fold transcript for each cell population reflective of 3 RT-PCR replicates). These results are in agreement with the fold-enrichment indicated by immunostaining for sarcomeric actins on equivalent cells cultured for 24 hours (n=2 biological replicates for each cell population). By the latter analysis, a mean of 7% of the unfractionated cells were cardiac, improving to 24% by Percoll fractionation (e.g. “fraction IV” cells) and further to 39% following immunodepletion on the Percoll-enriched cells.

Discussion

The results support the efficacy of selection of human ESC-derived cardiomyocytes using magnetic cell-sorting for appropriate cell surface markers. Of note, both positive and negative selection MACS protocols result in an acceptable cell yield: a mean recovery of 2.3% of the starting cell number for NCAM MACS positive selection (n=2) and a mean of 2.6% for anti-epithelial and fibroblastic cell MACS negative selection (n=3). Positive and negative selection strategies are not mutually exclusive; and procedures may involve a combination of the two.

The compositions and procedures provided in the description can be effectively modified by those skilled in the art without departing from the spirit of the invention embodied in the claims that follow.

1. A method of enriching for differentiating cells of interest, the method comprising:
partially dissociating an in vitro culture comprising human stem cells grown under differentiative conditions, to provide a population of cell aggregates;
selecting said cell aggregates for a phenotypic feature of interest present on said differentiating cells;
wherein an enriched cell population comprising differentiating cells of interest is obtained.

2. The method according to claim 1, wherein said selecting step comprises:
contacting said sample with a binding agent specific for a lineage specific marker present on said differentiating cells;
contacting said sample with a separation device; and
selecting for cell aggregates in the sample having binding agents bound thereto.

3. The method according to claim 2, wherein said binding agent specific for a lineage specific marker is an antibody.

4. The method according to claim 3, wherein said separation device comprises a magnetic separation device, and said antibody is coupled to a magnetic reagent.

5. The method according to claim 3, wherein said separation device comprises a particle sorter.

6. The method according to claim 1, wherein said human stem cells are grown as embryoid bodies.

7. The method according to claim 1, further comprising an additional step of:
enriching for said differentiating cells prior to said selecting step.

8. The method according to claim 7, wherein said enriching step comprises discontinuous density gradient centrifugation.

9. The method according to claim 7, wherein said enriching step comprises a negative selection for cells other than said differentiating cells of interest.

10. The method according to claim 9, wherein said cells other than said differentiating cells of interest comprise one or more of embryonic stem cells, fibroblasts and epithelial cells.

11. The method of claim 2, wherein said differentiating cells of interest comprise cells of the cardiomyocyte lineage.

12. The method according to claim 11, wherein said lineage specific marker is selected from the group consisting of NCAM (CD56); HNK-1; L-type calcium channels; cardiac sodium-calcium exchanger; Mlc2v; and Anf.

13. The method according to claim 1, wherein said cell aggregates comprise at least two and not more than about 50 cells.

14. The method according to claim 1, wherein at least about 50% of the total cells in said enriched cell population are said differentiating cells of interest.

15. An enriched cell population obtained by the method set forth in claim 1.

16. The enriched cell composition according to claim 15, wherein at least about 50% of the total cells in said enriched cell population are said differentiating cells of interest.

17. The enriched cell composition according to claim 15, wherein said cell aggregates comprise at least two and not more than about 50 cells.

18. The enriched cell composition according to claim 15, wherein said differentiating cells of interest comprise cells of the cardiomyocyte lineage.

19. The enriched cell composition according to claim 18, wherein said cells of the cardiomyocyte lineage express at least one of NCAM (CD56); HNK-1; L-type calcium channels; cardiac sodium-calcium exchanger; Mlc2v; and Anf.

20. The enriched cell composition according to claim 18, and a physiologically acceptable excipient.
